(19) United States
(12) Patent Application Publication
(10) Pub. No.: US 2026/0263546 A1
(43) Pub. Date: Sep. 10, 2026

(54) METHOD FOR ALLEVIATING DRY EYE SYNDROME USING SOYBEAN SEED ETHANOL DISTILLATE

(71) Applicant: BOTANICURE CO., LTD., Tainan City (TW)

(72) Inventors: I-Hung CHU, Tainan City (TW); Chih-Yi WENG, Tainan City (TW)

(21) Appl. No.: 19/535,071

(22) Filed: Feb. 10, 2026

Related U.S. Application Data
(63) Continuation-in-part of application No. PCT/CN2024/132295, filed on Nov. 15, 2024.
(60) Provisional application No. 63/599,449, filed on Nov. 15, 2023.

(51) Int. Cl. A61K 36/48; A61P 27/02

(52) U.S. Cl. CPC A61K 36/48; A61P 27/02; A61K 2236/333

(57) ABSTRACT

A method for alleviating dry eye syndrome includes administering to a subject in need thereof a composition containing a soybean seed ethanol distillate. The composition is a food product or a pharmaceutical composition. The pharmaceutical composition further contains a pharmaceutically acceptable carrier, and is formulated into a dosage form suitable for oral administration, topical administration, or parenteral administration.

### CROSS-REFERENCE TO RELATED APPLICATION

This application is a continuation-in-part (CIP) of International Application No. PCT/CN2024/132295, filed on Nov. 15, 2024, which claims priority to U.S. Provisional Patent Application No. 63/599,449, filed on Nov. 15, 2023. The aforesaid applications are incorporated by reference herein in their entirety.

### FIELD

The disclosure relates to a method for alleviating dry eye syndrome using a soybean seed ethanol distillate.

### BACKGROUND

Dry eye syndrome (DES) is mainly caused by insufficient tear secretion and excessive tear evaporation of an eye. The main symptoms of dry eye syndrome include dryness, redness, itching, photophobia, and pain. In severe cases, DES may even cause visual disturbance and ocular surface damage.

Artificial tears are mainly used clinically for treating mild dry eye syndrome, while anti-inflammatory drugs (AIDs) are required for treating severe dry eye syndrome. However, both the artificial tears and the anti-inflammatory drugs can only temporarily alleviate eye discomfort and are unable to increase tear secretion.

Glycine max (i.e., soybean) is a plant of the genus Glycine in the family Fabaceae, and a part of Glycine max that is mainly used is seeds thereof, which are considered a good source of protein. In TW I640318 B (corresponding to U.S. Ser. No. 10/543,243 B2), the applicant discloses an extract composition including a soybean seed extract, which is obtained by subjecting soybean seeds to an extraction treatment using water or an alcohol solution including alcohol at a concentration lower than 90 wt %, and a soybean seed vapor fraction, which is obtained by subjecting soybean seeds to a distillation treatment using water or an alcohol solution including alcohol at a concentration lower than about 15 wt %. The extract composition is proven to have the effects of promoting healing of a skin wound, enhancing nerve cell proliferation, and treating dementia and breast cancer. In TW I724417 B (corresponding to U.S. Ser. No. 11/083,766 B2), the applicant discloses use of the extract composition in alleviating pain and skin inflammation caused by cancer radiation therapy.

In spite of the aforesaid, there is still a need to develop a method that is effective in alleviation of dry eye syndrome.

### SUMMARY

Therefore, the present disclosure provides a method for alleviating dry eye syndrome, which can alleviate at least one of the drawbacks of the prior art, and which includes administering to a subject in need thereof a composition containing a soybean seed ethanol distillate.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 shows the tear-wetted length of Schirmer tear test strip determined in the mice of each group of Example 1, infra, in which the symbols and “**” represent p<0.05 and p<0.01, respectively (compared with the pathological control group), and the symbol “#” represents p<0.05 (compared with the comparative group).

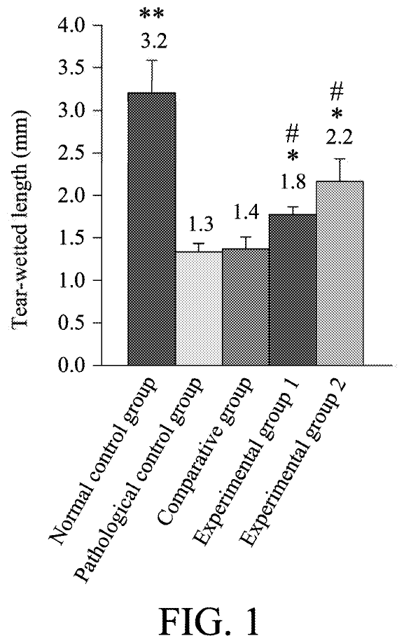

### DETAILED DESCRIPTION

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless otherwise defined, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

In the development of approaches to alleviate dry eye syndrome, the applicant surprisingly found that a soybean seed ethanol distillate can effectively increase tear secretion in mice with scopolamine-induced dry eye syndrome, and hence are expected to be effective in alleviation of dry eye syndrome.

Therefore, the present disclosure provides a method for alleviating dry eye syndrome, which includes administering to a subject in need thereof a composition containing a soybean seed ethanol distillate.

As used herein, the terms “dry eye syndrome”, “xerophthalmia”, “sclerophthalmia”, “keratoconjunctivitis sicca” and “dysfunctional tear syndrome” can be interchangeably used, and are intended to cover at least one of the following types: aqueous tear-deficient dry eye, mucin-deficient dry eye, lipid-deficient dry eye, and evaporative dry eye.

According to the present disclosure, the dry eye syndrome can be caused by various different factors, which may include, but are not limited to, deficiency in the tear-flow system, sleep disorder (such as insomnia and sleep apnea syndrome), natural aging process (particularly menopause), side effects of drugs (e.g., antidepressants, blood pressure medications, Parkinson's medications, antihistamines, and birth control pills), diseases that affects tear producing capability (such as Sjogren's syndrome, rheumatoid arthritis, and collagen vascular disease), wearing of contact lens, smoking, particulate matter (PM; e.g., PM 2.5), dry climate, insufficient blinking, and structural problems that prevent proper closure of an eyelid.

As used herein, the terms “administering” and “administration” can be interchangeably used, and refer to introducing, providing or delivering a pre-determined active ingredient to a subject by any suitable routes to perform its intended function.

As used herein, the term “subject” refers to any animal of interest, such as humans, monkeys, cows, sheep, horses, pigs, goats, dogs, cats, mice, and rats. In certain embodiments, the subject is a human.

As used herein, the terms “Glycine max”, “soybean”, and “vegetable soybean” can be interchangeably used, and are intended to cover soybeans that can be easily obtained by a person skilled in the art, or soybeans that can be collected from natural sources.

According to the present disclosure, the distillation treatment for producing the soybean seed ethanol distillate are within the expertise and routine skills of those skilled in the art. In this regard, reference may be made to, for example, TW I724417 B (corresponding to U.S. Ser. No. 11/083,766 B2) and TW I640318 B (corresponding to U.S. Ser. No. 11/452,753 B2, U.S. Pat. No. 11,400,128 B2, and U.S. Pat. No. 10,543,243 B2).

It can be understood that the procedures and parameter conditions for carrying out the distillation treatment for producing the soybean seed ethanol distillate may be varied depending on the processing method of soybean seeds, or an amount ratio of the soybean seeds to an ethanol solution, and so forth, so as to achieve the best distillation effect. That is to say, the operating conditions are within the expertise and routine skills of those skilled in the art.

According to the present disclosure, the distillation treatment for producing the soybean seed ethanol distillate may be performed using fresh soybean seeds, or soybean seeds that have been pre-processed by a treatment selected from the group consisting of a drying treatment, a grinding treatment, a cutting treatment, a pulverization treatment, and combinations thereof.

According to the present disclosure, the distillation treatment for producing the soybean seed ethanol distillate can be performed using the soybean seeds and the ethanol solution in a weight ratio ranging from 1:1 to 1:100. In an exemplary embodiment, the weight ratio of the soybean seeds to the ethanol solution is 1:10.

In certain embodiments, the soybean seed ethanol distillate is prepared by subjecting soybean seeds to the distillation treatment using an ethanol solution having an ethanol concentration ranging from 0.01 wt % to 15 wt %. In certain embodiments, the ethanol solution has an ethanol concentration ranging from 0.13 wt % to 15 wt %. In an exemplary embodiment, the ethanol solution has an ethanol concentration of 0.13 wt %.

According to the present disclosure, the distillation treatment for producing the soybean seed ethanol distillate may be carried out at a temperature ranging from 55° C. to 98° C. In an exemplary embodiment, the temperature is 90° C.

In certain embodiments, the composition containing the soybean seed ethanol distillate may be a food product.

In certain embodiments, the composition containing the soybean seed ethanol distillate may be a pharmaceutical composition.

According to the present disclosure, the pharmaceutical composition may be formulated into a dosage form suitable for parenteral administration, oral administration, or topical administration using technology well known to those skilled in the art.

According to the present disclosure, the pharmaceutical composition may further include a pharmaceutically acceptable carrier widely employed in the art of drug-manufacturing. For instance, the pharmaceutically acceptable carrier may include one or more of the following agents: solvents, buffers, emulsifiers, suspending agents, decomposers, disintegrating agents, dispersing agents, binding agents, excipients, stabilizing agents, chelating agents, diluents, gelling agents, preservatives, wetting agents, lubricants, absorption delaying agents, liposomes, thickeners, and the like. The choice and amount of the aforesaid agents are within the expertise and routine skills of those skilled in the art.

For parenteral administration, the pharmaceutical composition according to the present disclosure may be formulated into an injection, e.g., a sterile aqueous solution or a dispersion, using technology well known to those skilled in the art.

The pharmaceutical composition according to the present disclosure may be administered via one of the following parenteral routes: intraperitoneal injection, intrapleural injection, intramuscular injection, intravenous injection, intraarterial injection, intraarticular injection, intrasynovial injection, intrathecal injection, intracranial injection, intraepidermal injection, subcutaneous injection, intradermal injection, intralesional injection, and sublingual administration.

According to the present disclosure, the dosage form suitable for oral administration includes, but is not limited to, sterile powders, tablets, troches, lozenges, pellets, capsules, dispersible powders or granules, solutions, suspensions, emulsions, syrup, elixir, slurry, and the like.

According to the present disclosure, the pharmaceutical composition may be formulated into an external preparation suitable for topical application to the skin using technology well known to those skilled in the art. The external preparation includes, but is not limited to, emulsions, gels, ointments, creams, patches, liniments, powder, aerosols, sprays, lotions, serums, pastes, foams, drops, suspensions, salves, and bandages.

According to the present disclosure, the external preparation may be produced by mixing the above-mentioned pharmaceutical composition with a base well known to those skilled in the art.

According to the disclosure, the base may include one or more of the followings additives: water, alcohols, glycols, hydrocarbons (such as petroleum jelly and white petrolatum), waxes (such as paraffin and yellow wax), preserving agents, antioxidants, surfactants, absorption enhancers, stabilizing agents, gelling agents (such as Carbopol® 941, microcrystalline cellulose, and carboxymethyl cellulose), active agents, humectants, odor absorbers, fragrances, pH adjusting agents, chelating agents, emulsifiers, occlusive agents, emollients, solubilizing agents, penetration enhancers, thickeners, anti-irritants, preservatives, colorants, propellants, and so forth. The choice and amount of the aforesaid additives are within the expertise and routine skills of those skilled in the art.

In certain embodiments, the pharmaceutical composition is an external preparation containing a thickener present in an amount ranging from 1 wt % to 20 wt % and a preservative present in an amount ranging from 0.1 wt % to 10 wt %. In an exemplary embodiment, the pharmaceutical composition is an ointment or a gel containing a thickener present in an amount of 9.65 wt % and a preservative present in an amount of 0.35 wt %.

In some embodiments, the aforesaid thickener may include polyacrylic acid (carbomer), glyceryl polyacrylate, glyceryl polymethacrylate, sodium hyaluronate, and hyaluronic acid.

In still some embodiments, the aforesaid preservative may be selected from the group consisting of benzyl alcohol, chloroxylenol, and a combination thereof.

According to the present disclosure, the dose and frequency of administration of the composition containing the soybean seed ethanol distillate may vary depending on the following factors: the severity of the illness or disorder to be treated, routes of administration, and age, physical condition and response of the subject to be treated. In general, the composition may be administered in a single dose or in several doses. For example, the composition may be administered twice a day.

The disclosure will be further described by way of the following examples. However, it should be understood that the following examples are solely intended for the purpose of illustration and should not be construed as limiting the disclosure in practice.

### EXAMPLES

### Example 1. Evaluation of Efficacy of Soybean Seed Ethanol Distillate in Alleviation of Dry Eye Syndrome

### Experimental Materials:

### 1. Preparation of Soybean Seed Ethanol Distillate:

The soybean seed ethanol extract and the soybean seed ethanol distillate used in the following examples were prepared according to the method described in TW I640318 B (corresponding to U.S. Ser. No. 10/543,243 B2).

Briefly, a powder of soybean (Glycine max) seeds harvested from Tainan City was mixed with an ethanol solution having an ethanol concentration of 0.13 wt % in a weight ratio of 1:10, followed by conducting a distillation treatment at 90° C. for a time period, and the condensates thus formed were collected, thereby obtaining the soybean seed ethanol distillate.

### 2. Preparation of Gel Containing Soybean Seed Ethanol Distillate:

Each of Gels 1 to 3 used in this example was prepared using the recipe shown in Table 1 below. The Gel 1 was prepared according to the following steps. Initially, only water was heated to a temperature ranging from 95° C. to 100° C. (i.e., no soybean seed ethanol distillate was used). Thereafter, polyacrylic acid (carbomer), glyceryl polymethacrylate, and sodium hyaluronate were added to the water that was heated, followed by stirring, thereby obtaining a first mixture. After the first mixture was cooled, benzyl alcohol and chloroxylenol were added thereto so as to obtain the Gel 1. The Gel 2 was prepared according to the following steps. First, the soybean seed ethanol distillate obtained in Section 1 of this example was dissolved in water, and then heated to a temperature ranging from 95° C. to 100° C., so as to obtain a second mixture. Next, polyacrylic acid (carbomer), glyceryl polymethacrylate, and sodium hyaluronate were added to the second mixture, followed by stirring, thereby obtaining a third mixture. After the third mixture was cooled, benzyl alcohol and chloroxylenol were added thereto so as to obtain the Gel 2. The Gel 3 were prepared according to the following steps. First, the soybean seed ethanol distillate obtained in Section 1 of this example was solely heated to a temperature ranging from 95° C. to 100° C. (i.e., no water was used), and then polyacrylic acid (carbomer), glyceryl polymethacrylate, and sodium hyaluronate were added thereto, followed by stirring, thereby obtaining a fourth mixture. After the fourth mixture was cooled, benzyl alcohol and chloroxylenol were added thereto so as to obtain the Gel 3.

TABLE 1

| | Gel 1 | Gel 2 | Gel 3 |
|---|---|---|---|
| Ingredient | Amount (wt %) | | |
| Soybean seed ethanol | 0.00 | 20.00 | 90.00 |
| distillate | | | |
| Polyacrylic acid | 0.15 | 0.15 | 0.15 |
| (carbomer) | | | |
| Glyceryl | 10.00 | 9.99 | 9.49 |
| polymethacrylate | | | |
| Sodium hyaluronate | 0.01 | 0.01 | 0.01 |
| Benzyl alcohol | 0.28 | 0.28 | 0.28 |
| Chloroxylenol | 0.07 | 0.07 | 0.07 |
| Water | 89.49 | 69.50 | 0.00 |

### 3. Experimental Mice:

Male C57BL/6 mice (8 weeks old; with a body weight of approximately 24 g) used in the following experiments were purchased from BioLasco Taiwan Co., Ltd. All the experimental mice were housed in an animal room with an independent air conditioning system under the following laboratory conditions: an alternating 12-hour light and 12-hour dark cycle, a temperature maintained at 24° C. to 26° C., and a relative humidity maintained at 55% to 65%. Furthermore, water and feed were provided ad libitum for all the experimental mice. All experimental procedures involving the experimental mice were in compliance with the animal management standards of the Association for Assessment and Accreditation of Laboratory Animal Care, International.

### Experimental Procedures:

### A. Induction of Dry Eye Syndrome:

First, the mice were randomly divided into 5 groups, including a normal control group (n=4), a pathological control group (n=5), a comparative group (n=4), and two experimental groups (i.e., experimental groups 1 (n=5) and 2 (n=4)). Next, the mice in each of the pathological control group, the comparative group, and the experimental groups 1 and 2 were subjected to subcutaneous injection with scopolamine (Sigma; Cat. No. S0929) (dose: 0.5 mg/mouse) (formulated in 0.2 mL of normal saline) twice a day for a time period of 21 days, so as to induce dry eye syndrome in the mice. The mice in the normal control group received no treatment.

### B. Administration of Gel 1, 2 or 3:

While being subjected to the subcutaneous injection with the scopolamine, the mice in the comparative group and the experimental groups 1 and 2 were also respectively administered with the Gels 1 to 3, where the Gel 1, 2 or 3 was applied to the skin around the eyes of the mice of a corresponding group (dose: 50 mg/cm²), twice a day for the time period of 21 days. The mice in the normal control group and the pathological control group received no treatment.

### C. Schirmer's Tear Test:

On the final day of the time period of 21 days described above, the mice in each group were subjected to Schirmer's tear test using Schirmer tear test strips (size: 1 mm×40 mm; purchased from OPTITECH CORP.). In the Schirmer's tear test, each of the Schirmer tear test strips was placed in a right lower eyelid of a respective one of the mice in each group for 30 seconds, so as to measure the tear-wetted length in each group.

The experimental data thus obtained are expressed as mean±standard error of the mean (SEM), and were analyzed using Student's t-test, so as to evaluate the differences between the groups. Statistical significance is indicated by p<0.05. The results were shown in the FIGURE.

### Results:

Referring to the FIGURE, the tear-wetted length determined in the pathological control groups was significantly lower than that determined in the normal control group, indicating that the scopolamine successfully induced dry eye syndrome and caused insufficient tear secretion in the mice of the pathological control groups. In addition, compared with the pathological control group or the comparative group, the tear-wetted length determined in each of the experimental groups 1 and 2 was significantly increased, and the higher the concentration of the soybean seed ethanol distillate is, the greater the increase in the tear-wetted length is. These results show that the soybean seed ethanol distillate can enhance tear secretion in a dose-related manner, and hence is expected to have a high potential for use in preparation of a composition for alleviating dry eye syndrome.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, FIGURE, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for alleviating dry eye syndrome, comprising administering to a subject in need thereof a composition containing a soybean seed ethanol distillate.

2. The method as claimed in claim 1, wherein the soybean seed ethanol distillate is prepared by subjecting soybean seeds to a distillation treatment using an ethanol solution having an ethanol concentration ranging from 0.01 wt % to 15 wt %.

3. The method as claimed in claim 2, wherein the ethanol solution has an ethanol concentration ranging from 0.13 wt % to 15 wt %.

4. The method as claimed in claim 1, wherein the composition is a food product.

5. The method as claimed in claim 1, wherein the composition is a pharmaceutical composition.

6. The method as claimed in claim 5, wherein the pharmaceutical composition further contains a pharmaceutically acceptable carrier.

7. The method as claimed in claim 5, wherein the pharmaceutical composition is in a dosage form selected from the group consisting of an oral dosage form, a topical dosage form, and a parenteral dosage form.
